(19) United States
(12) Patent Application Publication
(10) Pub. No.: US 2013/0197338 A1
(43) Pub. Date: Aug. 1, 2013

(54) PAIN SIGNAL MEASUREMENT DEVICE AND PAIN SIGNAL MEASURING AND CONTROLLING METHOD THEREOF

(71) Applicant: Electronics and Telecommunications Research Institute, Daejeon (KR)

(72) Inventors: Han Young YU, Daejeon (KR); Yark Yeon KIM, Daejeon (KR); Yong Ju YUN, Daejeon (KR); Won Ick JANG, Daejeon (KR)

(73) Assignee: Electronics and Telecommunications Research Institute, Daejeon (KR)

(21) Appl. No.: 13/744,324

(22) Filed: Jan. 17, 2013

(30) Foreign Application Priority Data
Jan. 30, 2012 (KR) 10-2012-0009018
Sep. 7, 2012 (KR) 10-2012-0099448

(51) Int. Cl. A61N 1/36; A61B 5/04

(52) U.S. Cl. CPC A61N 1/36021; A61B 5/04
USPC 600/377; 607/46

(57) ABSTRACT

Provided is a pain signal measurement device including a microprobe array inserted into a skin to measure a pain signal. The microprobe array includes a guard electrode disposed on a substrate; a plurality of microprobes penetrating the substrate and the guard electrode, electrically insulated from the guard electrode, and measuring a voltage or current of the skin into which the microprobe array is inserted; and an insulating layer disposed between the guard electrode and a guarded electrode of each of the microprobes to reduce a noise between the microprobes. A surface of the insulating layer of each of the microprobes is grounded to the guard electrode.

### CROSS-REFERENCE TO RELATED APPLICATIONS

This US non-provisional patent application claims priority under 35 USC §119 to Korean Patent Application No. 10-2012-0009018, filed on Jan. 30, 2012, and Korean Patent Application No. 10-2012-0099448, filed on Sep. 7, 2012, the entirety of which is hereby incorporated by reference.

### BACKGROUND OF THE INVENTION

The present general inventive concept relates to pain signal measurement devices and pain signal measuring and controlling methods thereof

The inventive concept provides a technique capable of quantitatively classifying pains which have been classified by means of a method for qualitatively classifying pains, i.e., acute and chronic pains that persons feel. In general, the degree and feeling of pains are different according to persons that feel the pains. For this reason, aspects of pains manifested from the brain are also different from each other. Since the degree of pains has become qualitative, it is difficult to accurately understand diseases. Accordingly, the inventive concept is concerned with a technique for quantifying qualified pains by measuring a pain signal among biosignals. That is, the inventive concept is concerned with a technique for quantifying pains through three-dimensional images by measuring a pain signal, among biosignals, at a dorsal root ganglion via a microprobe array and deriving physical factors from the measured pain signal.

When there is a lesion at the outside, neurotransmitters are released due to the lesion, an action potential fires at a peripheral nerve. The action potential is transmitted to a spinal cord via nerves distributed throughout the body. A pain signal transmitted to the spinal cord is transmitted to a brain. Through the steps of sensing and manifesting a pain at the brain, the pain is felt.

Pain is defined as “an unpleasant sensory and emotional experience associated with actual or potential tissue damage or describe in terms of such damage”. It seems that the definition of pain is established because a pain signal is transmitted to a brain through nerves to individually define the overall flow manifested at the brain. Namely, the definition of pain means that a pain is a signal felt at the body and the intensity of the pain varies depending on the degree manifested at the brain. The manifestation of pain means that neurotransmitters varying in amount depending on persons are released to show the intensity of pain that varies depending on the persons.

Accordingly, it has been reported that it is difficult to quantify the degree and intensity of pain. For this reason, the pain has been regarded as an empirical value.

However, occurrence of pain and path transmission through nerves show the common transmission signal of all the persons. That is, generation and transmission of an electrochemical signal caused by occurrence of pain are characterized in that the intensity of pain varies depending on the persons but the flow of the pain and the signal transmission are commonly revealed. Thus, the same type of signal is commonly transmitted to a brain before a pain signal is manifested at the brain and modulated with different neurotransmitters. This means that if a pain signal is measured before its modulation at a brain, the pain may be quantified.

When a pain signal is generated at a peripheral nerve and transmitted to the central nerve, all pain signals pass through dorsal root ganglions along ascending neural circuits. The dorsal root ganglion may be a synapse that is not distributed at a deep place of the spinal cord but exists at a front portion introduced into the spinal cord. The dorsal root ganglion may be a point to solve the problem that a pain signal cannot be measured on the way because most neural circuits are transmitted to the spinal cord and brain while being covered with muscles and bones when the pain signal is generated and transmitted. That is, the pain signal reaching the spinal cord suffers from various problems caused by the muscles and the bones when the pain signal is measured. However, since many parts of the dorsal root ganglion are exposed, the dorsal root ganglion may be the most suitable place for measuring the pain signal.

There is still a problem to be solved for measuring a pain signal under the assumption that the pain signal is introduced into a dorsal root ganglion to be transmitted to the spinal cord and brain. The problem is that it is necessary to overcome high contact resistance of a skin when the pain signal is measured through the skin. That is, although the skin contains moisture, it has too high contact resistance to measure an inner neural signal through the skin.

The problem means the disadvantage that the neural signal cannot be measured accurately because a voltage applied to the skin is measured high due to contact resistance and a practical neural signal remains at the level of noise when the neural signal is measured through an external electrode. That is, the typical skin is classified into the epidermis having a thickness above about 70 micrometers and the dermis disposed below the epidermis.

Since ions are rarely distributed at the epidermis, contact resistance of the epidermis is significantly high. Since the dermis includes a number of ions therein, contact resistance of the dermis is low.

Accordingly, in order to measure a signal of a practical neural circuit, use of a probe having a length greater than a thickness of the epidermis may be one of the methods for accurately measuring a pain signal. Moreover, it is necessary to overcome another problem that a new pain is aroused to measure a pain signal. That is, since a new pain is manifested when a typical probe reaches the dermis through the epidermis to measure a pain signal using the probe, a pain cannot be measured accurately. Thus, the shape of a probe for measuring a pain signal must be limited in size to prevent production of pain.

### SUMMARY OF THE INVENTION

An aspect of the inventive concept provides a pain signal measurement device. The pain signal measurement method may include a microprobe array inserted into a skin to measure a pain signal. The microprobe array may include a guard electrode disposed on a substrate; a plurality of microprobes penetrating the substrate and the guard electrode, electrically insulated from the guard electrode, and measuring a voltage or current of the skin into which the microprobe array is inserted; and an insulating layer disposed between the guard electrode and a guarded electrode of each of the microprobes to reduce a noise between the microprobes. A surface of the insulating layer of each of the microprobes may be grounded to the guard electrode.

Another aspect of the inventive concept provides a pain signal measurement method of a pain signal measurement device including at least one microprobe array which includes a guard electrode disposed on a substrate; a plurality of microprobes penetrating the substrate and the guard electrode and electrically insulated from the guard electrode; and an insulating layer disposed between the guard electrode and a guarded electrode of each of the microprobes to reduce a noise between the microprobes, wherein a surface of the insulating layer of each of the microprobes is grounded to the guard electrode to be guarded by the insulating layer of each of the microprobes and the guard electrode. The pain signal measurement method may include attaching the at least one microprobe array to the skin; and making the at least one microprobe array penetrate the epidermis of the skin to measure an action potential of nerves distributed at the dermis of the skin.

Further another aspect of the inventive concept provides a pain control method using a pain signal measurement device including at least one microprobe array which includes a guard electrode disposed on a substrate; a plurality of microprobes penetrating the substrate and the guard electrode and electrically insulated from the guard electrode; and an insulating layer disposed between the guard electrode and a guarded electrode of each of the microprobes to reduce a noise between the microprobes, wherein a surface of the insulating layer of each of the microprobes is grounded to the guard electrode to be guarded by the insulating layer of each of the microprobes and the guard electrode. The pain control method may include making the least one microprobe array penetrate the epidermis of the skin to measure a pain signal from nerves distributed at the dermis of the skin; transmitting the measured pain signal to an external circuit; and making the external circuit transmit, to the at least one microprobe array, a pain control signal for modulating the pain signal based on the transmitted pain signal when the pain signal is transmitted to a brain.

### BRIEF DESCRIPTION OF THE DRAWINGS

The inventive concept will become more apparent in view of the attached drawings and accompanying detailed description. The embodiments depicted therein are provided by way of example, not by way of limitation, wherein like reference numerals refer to the same or similar elements. The drawings are not necessarily to scale, emphasis instead being placed upon illustrating aspects of the inventive concept.

FIG. 1 illustrates a microprobe array for measuring a pain signal.

FIG. 2 shows that a microprobe array is inserted into the skin.

FIG. 3 illustrates an apparatus for attaching an electrode to the skin using a suction.

FIG. 4 illustrates a microprobe array attached to measure and control a pain signal at a dorsal root ganglion.

FIG. 5 shows the distribution of a microprobe array signal to measurement of a pain signal.

FIG. 6 illustrates derivation of physical agents according to analysis of a pain signal.

FIG. 7 illustrates mapping of a pain from physical agents of a pain signal.

### DETAILED DESCRIPTION

Embodiments of the inventive concept will now be described with reference to the accompanying drawings. However, it should be noted that the inventive concept may be variously embodied and is not limited to only the illustrated embodiments. Rather, the illustrated embodiments are presented as teaching examples. Throughout the written description and drawings, like reference numbers and labels are used to indicate like or similar elements.

According to the inventive concept, a pain may be quantified by a painless microprobe for measuring a pain signal, measurement of a pain signal at a dorsal root ganglion for accurately measuring of the pain signal, deriving physical factors capable of deciding the intensity of pain from the measured pain signal, and a three-dimensional mapping technique for quantifying pains depending on the types, intensity, and positions of pains through the physical agents.

A pain signal measurement device and a pain signal measurement method according to embodiments of the inventive concept may roughly include a microprobe for measuring a pain signal, a position method at a dorsal root ganglion for measuring the pain signal of the microprobe, contact of the body with a pain signal measurement probe through a suction-type vacuum device to accurately measure a pain signal, a method for measuring the entire pain signal of the body by locating a plurality of pain signal measurement probes at a dorsal root ganglion to measure the entire pain signal of the body, a method for measuring a voltage generated from a pain signal depending on electrode distribution through a plurality of electrodes for measuring a pain signal, a method for deriving physical factors from a pain distribution signal, and a three-dimensional matrix based three-dimensional mapping technique for quantifying pains depending on physical factors and the types and positions of pain signals based on the physical factors.

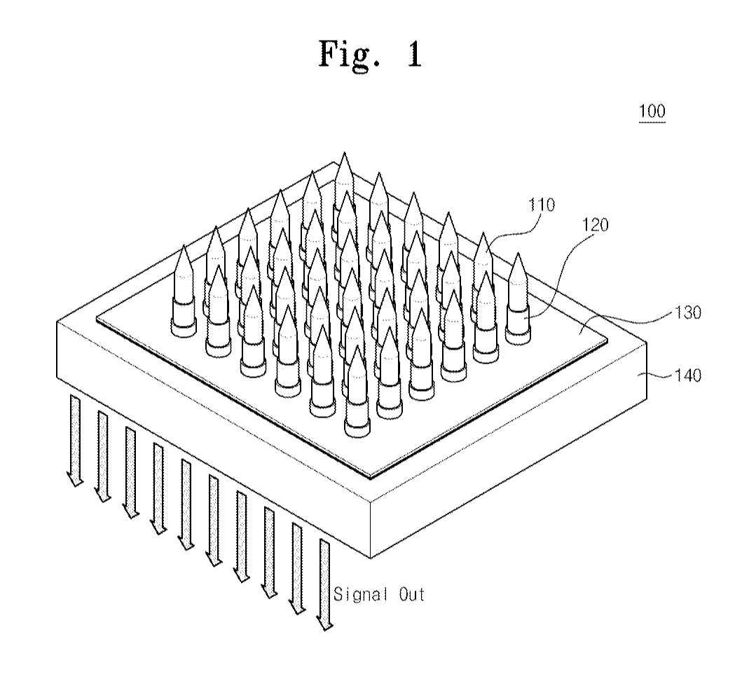

FIG. 1 illustrates a microprobe array according to an embodiment of the inventive concept. Referring to FIG. 1, a microprobe array 100 for measuring a pain signal includes guarded electrodes 110, insulating layers 120, a guard electrode 130, and a substrate 140.

The guarded electrodes 110 are disposed at the end of each microprobe to measure a pain signal. Each microprobe penetrates the substrate 140 and the guard electrode 130 and is electrically insulated from the guard electrode 130. Each microprobe may measure a voltage or current of a microprobe-inserted skin. The guard electrode 130 may be disposed on a substrate. The guard electrode 130 may be in the form of continuous or discontinuous film and may be grounded solely or commonly.

The insulating layer 120 may be disposed between a guard electrode 130 and a guarded electrode 110 of each microprobe to reduce a noise caused by coupling between microprobes, a noise caused by an external electric field, and a noise included in an electrical signal of the dermis of a target skin. A surface of an insulating layer of each microprobe may be grounded to the guard electrode 130.

A typical microprobe array includes only a probe electrode for measuring a pain signal. That is, since an insulating layer or the like for reducing a noise is not provided between electrodes, the typical microprobe array includes only a space in which a sample is placed. Even the configuration of the typical microprobe array makes it possible to easily measure a signal because the intensity of the signal has a much greater value than that of a noise when a voltage or current measured by a probe electrode is high. However, when a voltage or current desired to be measured is low, i.e., a signal-to-noise ratio is low, a high signal cannot be measured due to a noise generated between measurement probes. This leads to the disadvantage that the measurement probes form one noise loop to cause a new noise. Accordingly, fabrication of an electrode capable of minimizing the noise must be accompanied with measurement of a low voltage and low current.

In the meantime, the microprobe array 100 according to the inventive concept may overcome the above disadvantage through fabrication of an electrode including guarded electrodes 110, a guard electrode 130, and insulating layers 120, as shown in FIG. 1. That is, generation of the noise may be minimized by providing an insulating layers 120 between measurement probes and forming a guard electrode 130 capable of solely or commonly grounding surfaces of the insulating layers 120.

In addition, a noise introduced from an electrode that does not perform measurement is grounded to be removed. Thus, a pain signal may be practically measured without noise. Each of the guard electrodes 130 may uniformly establish and measure an electric field desired to be measured at each of the guard electrode 130 to measure a voltage of constant value. In other words, a non-uniform electric field established according to the structure of an electrode is grounded to be removed at the guard electrode 130, which serves to minimize the edge effect occurring at an AC electric field.

In an exemplary embodiment, a microprobe for measuring a pain signal may include commonly grounded guard electrodes 130. The guard electrodes 130 may be separated from each other to be grounded commonly or solely (not shown).

In an exemplary embodiment, a microprobe for measuring a pain signal may have a diameter less than 100 micrometers so as not to cause a pain and a length above 70 micrometers that is greater than a thickness of the epidermis of a skin.

In an exemplary embodiment, a distance between electrodes of a microprobe array may be shorter than Myelin sheath length (1 millimeter) in a neural circuit.

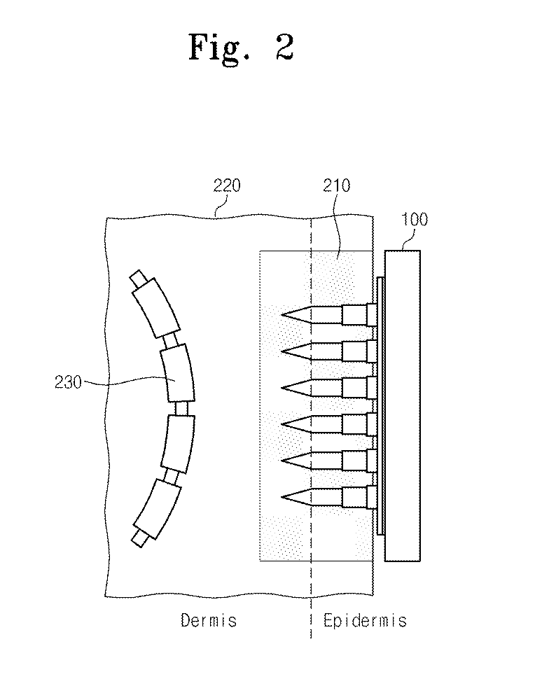

FIG. 2 illustrates a pain signal measurement method using the microprobe array 100 shown in FIG. 1. Referring to FIG. 2, depth of the microprobe array 100 is great enough to penetrate the epidermis 210 such that conductivity is improved by ions formed at the dermis 220. The microprobe array 100 may measure an action potential flowing along nerves 230.

In general, the conductivity is high at the dermis 210 because a number of electrolytes enhancing electroconductivity are distributed at the dermis 210. Since a length of a probe of the microprobe array 100 is greater than a thickness of the dermis 220 to overcome low conductivity at the epidermis 210, an electrical signal at the dermis 220 may be sufficiently read as a low-noise signal. The microprobe array 100 may be formed with a length that is smaller than Myelin sheath distance (1 millimeter) to read a signal at a practical neuron.

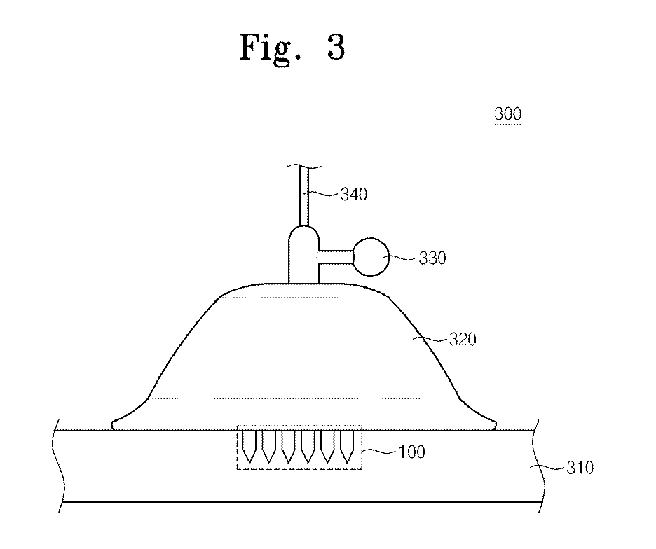

FIG. 3 illustrates a method for attaching the microprobe array 100 shown in FIG. 1 to the skin. Referring to FIG. 3, the microprobe array 100 may be tightly attached to a surface of the skin 310 by using a suction-type attaching device 330. The attaching device 330 removes the air of a suction 320 to lower a pressure and thus may attaches the microprobe array 100 to the skin 310. The microprobe array 100 penetrates the epidermis and contacts electrolytes to increase a signal-to-noise ratio of low resistance, i.e., high conductivity and thus may measure a signal. The measured signal is introduced or transmitted through a signal transmission line 340.

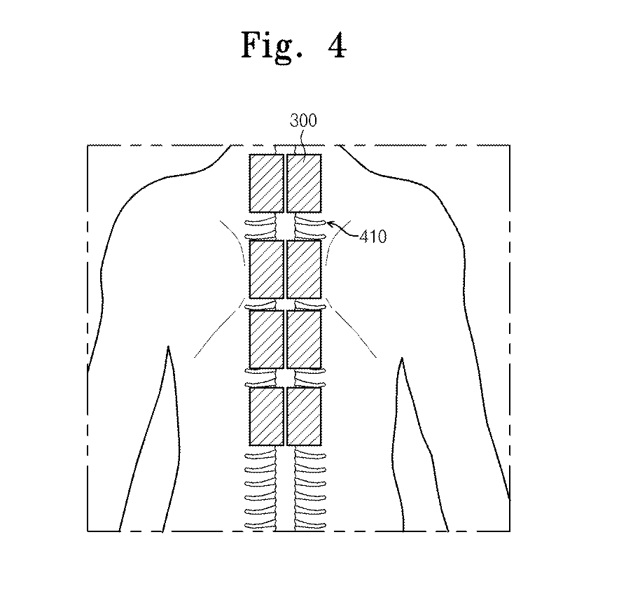

FIG. 4 illustrates a pain signal measurement method for measuring a pain signal at a dorsal root ganglion 410 using a pain signal measurement device according to an embodiment of the inventive concept. Referring to FIG. 4, a pain signal measurement device 300 including a plurality of microprobe arrays (e.g., microprobe array 100 in FIG. 1) may measure a pain signal at the dorsal root ganglion 410. The dorsal root ganglion 410 is distributed at an outer portion of the spine. Since nerves are distributed to be closest to the outside of the dorsal root ganglion 410, the dorsal root ganglion 410 acts as a body part where a pain signal can be measured best. By attaching the microprobe arrays 100 to the pain signal measurement part, pain signals introduced from respective parts of the body may be measured through the microprobe arrays 100.

By attaching the microprobe array 100 to arms and legs, a reference signal having a different type from a pain signal is generated to act as a pain measuring reference that is different from a pain signal caused by a pain (not shown). That is, if a pain signal is transmitted from the leg, a microprobe array 100 for biosignal modulation is attached to a part, which is similar to a pain-made part, to generate a new type signal and thus the new type signal may be measured at the dorsal root ganglion 410. Speed and position of a pain signal transmitted from a pain-made part are estimated to establish a reference for deciding generation type and speed of the pain signal.

From the reference, a reference point for other pain signals may be applied to reversely track not only the position of a pain but also a pain at an associated organ of the human body. In addition, a new reference point is established to find out a position of cause of a pain such as a phantom pain that occurs without an organ.

Returning to FIG. 4, a pain signal may be controlled using the microprobe array 100. An electrode of the microprobe array 100 is a measurement base to measure a pain signal. Under this standpoint, a pain signal may be a reference point to transfer its size and shape to an external circuit through the microprobe array 100. This means that a pain signal appears on the outside. If reversely interpreted, this means that a new signal may be introduced to the inside.

That is, when a pain occurs and a pain signal is transmitted to the brain and if a pain control signal modulating the pain signal is reversely introduced from the outside, the pain signal appears as a new type signal at the brain. That is, the pain signal may be removed by periodically introducing the pain control signal through modulation of a phase or a signal having ±90 degrees to the pain signal. Alternatively, by periodically introducing a signal having an earlier cycle than the pain signal, the pain signal is made so as not be distinguished from the introduced signal and thus a pain control signal may be introduced to prevent the brain from feeling a pain.

A pain control signal capable of controlling a pain signal may be an AC signal or a DC signal. In order to control a pain signal, the pain control signal may control a pain by adjusting a phase difference at the outside. The controlled and modulated external input signal may be a sine wave, a pulse wave, a square wave or a combination thereof.

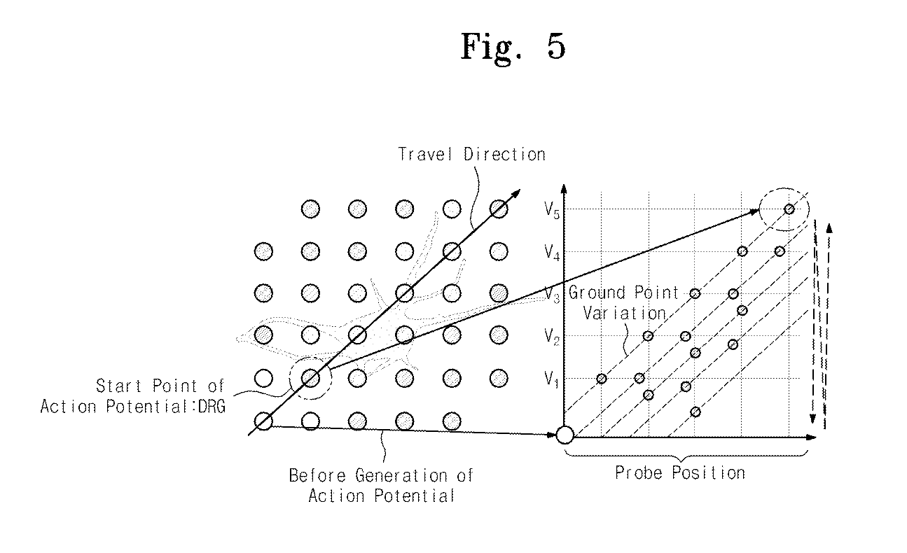

FIG. 5 illustrates a pain signal analysis method using the microprobe array 100 shown in FIG. 1. Referring to FIG. 5, when a pain signal is introduced to a dorsal root ganglion, there is a potential start point at the microprobe array 100. That is, a pain signal is not measured at electrodes before generation of an action potential but measured at a start point of the action potential, and the action potential is additionally generated at the other electrodes of travel direction.

Accordingly, the intensity of an action potential is high at a dorsal root ganglion where an action potential is generated first and the intensity of an action potential varies depending on positions of probes. That is, an action potential exhibits AC characteristics while its vibration is greatest at a part where the action potential is generated first and vibrates with the amplitude having a low voltage at a part that is not a travel direction of the action potential.

The intensity of the pain signal also varies depending on the arrangement of the microprobe array 100 and variation of a potential. That is, if six adjacent electrodes are differently grounded, an action potential having a highest signal appears differently according to the respective grounded shapes of the electrodes. Likewise, the arrangement of the microprobe array 100 and the grounded shapes of the electrodes are made differently to measure a high-sensitivity signal to a pain signal. In addition, if the pain signal is measured under the above distribution according to time (not shown), a travel direction and an introduction direction of pain may be estimated. This is a reference for understanding the cause of a pain.

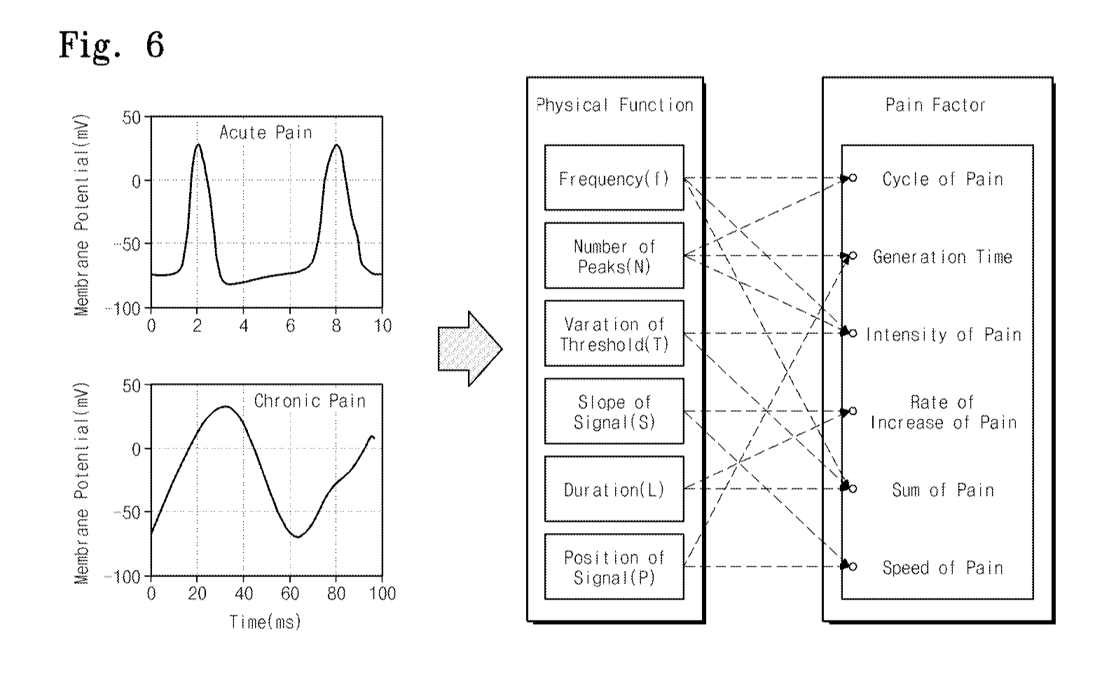

FIG. 6 illustrates a method for deriving physical factors from a pain signal according to an embodiment of the inventive concept. Referring to FIG. 6, a pain signal may be roughly classified into an acute pain signal and a chronic pain signal. The classification is dependent on the propagation type of a pain signal generated. That is, in case of an acute pain, if a potential rapidly increases and is felt as a pain when the potential exceeds a threshold, the pain is propagated well at Aδ fibers among nerve fibers. Meanwhile, a chronic pain is low in propagation speed. However, duration of the chronic pain is longer than that of the acute pain.

The chronic pain is transferred through C nerve fibers among the nerve fibers. Thus, physical factors distinguishing and deciding pains may define physical parameters for occurrence of pain through a frequency of a pain signal detected by a pain signal, the number of peaks of the pain signal, variation of a threshold of the pain signal, increasing and decreasing slopes of the pain signal, duration of the pain signal, a microprobe of the pain signal, and a position of the pain signal at the modulated signal.

The physical factors have a close relation to a cycle of a pain, occurrence time of the pain, the intensity of the pain, a rate of increase of the pain, the sum of pains, and speed of the pain. The physical factors mean patterns of pains that persons have. For example, an acute pain is characterized in high frequency, a large number of peaks, a high slope, but short duration.

A chronic pain is characterized in long duration, which is different according to diseases of pains and persons. However, pain variations depending on persons are similar in pattern while values of pains that the persons feel are different.

Accordingly, if a correlation of the physical function is visualized, the visualized correlation is a reference for putting a pain into shape.

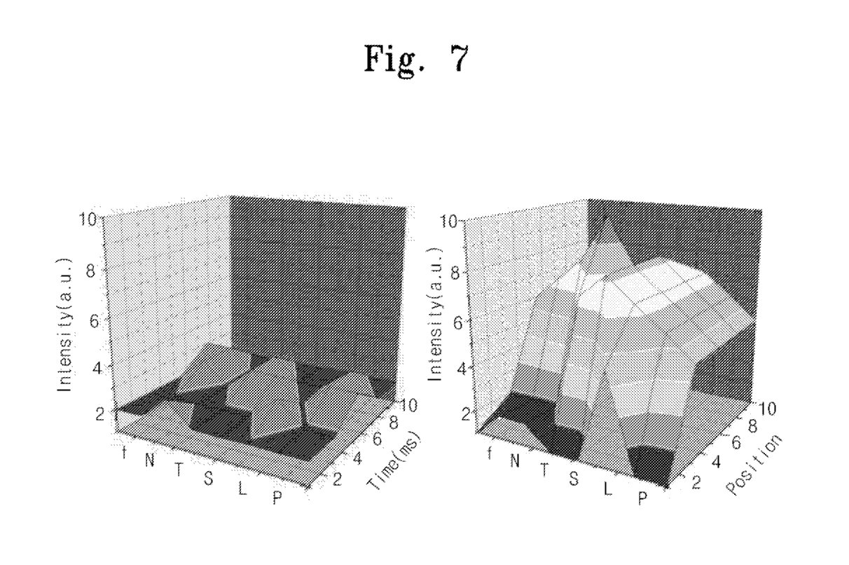

FIG. 7 illustrates a three-dimensional mapping technique of a pain signal according to an embodiment of the inventive concept. Referring to FIG. 7, physical agents for pain manifestation varies depending on a frequency of a pain signal, the number of peaks of the pain signal, variation of threshold at the pain signal, increasing and decreasing slopes of the pain signal, duration of the pain signal, a microprobe of the pain signal, and a position of the pain signal at a modulated signal. The signal intensity forms one pattern, and the pain signal may be mapped from the pattern. That is, an acute pain and various chronic pains may be mapped. As shown in FIG. 7, the pain signal has different shapes, which vary depending on pain classification, relative to the respective physical agents and the axis of time. Thus, a reference is established for three-dimensionally quantifying a pain.

Referring to FIG. 7, if a pain occurs in a left state where a pain did not occur, a state transitions to a new state, i.e., right state. Images displayed according to pains may be mapped by analyzing the shape of the transition, i.e., three-dimensional shape.

According to the above-described pain signal measurement device and method, a pain signal transmitted to a brain can be accurately measured to measure the intensity of a pain. In addition, a reference for classifying pains including acute and chronic pains can be established relative to different positions of pains that persons have and acute and chronic pains. In addition, a pain can be indexed and quantified to establish a reference for diagnosing primary diseases.

While the inventive concept has been particularly shown and described with reference to exemplary embodiments thereof, it will be apparent to those of ordinary skill in the art that various changes in form and detail may be made therein without departing from the spirit and scope of the inventive concept as defined by the following claims.

What is claimed is:

1. A pain signal measurement device comprising:
a microprobe array inserted into a skin to measure a pain signal,
wherein the microprobe array comprises:
a guard electrode disposed on a substrate;
a plurality of microprobes penetrating the substrate and the guard electrode, electrically insulated from the guard electrode, and measuring a voltage or current of the skin into which the microprobe array is inserted; and
an insulating layer disposed between the guard electrode and a guarded electrode of each of the microprobes to reduce a noise between the microprobes,
wherein a surface of the insulating layer of each of the microprobes is grounded to the guard electrode.

2. The pain signal measurement device as set forth in claim 1, which is solely grounded to the guard electrode disposed on the surface of the insulating layer of each of the microprobes.

3. The pain signal measurement device as set forth in claim 1, which is commonly grounded to the guard electrode disposed on the surface of the insulating layer of each of the microprobes.

4. The pain signal measurement device as set forth in claim 1, further comprising:
a substrate electrode setting a surface voltage of the microprobe electrode and the guard electrode as a reference voltage.

5. The pain signal measurement device as set forth in claim 1, wherein each of the microprobes has a length of 100 micrometers or less and a length of 50 micrometers or more.

6. The pain signal measurement device as set forth in claim 1, wherein a distance between guarded electrodes at the respective microprobes is shorter than Myelin sheath length.

7. The pain signal measurement device as set forth in claim 1, further comprising:
a signal transmission line receiving a pain signal from a guarded electrode of each of the microprobes or transmitting a pain control signal to the guarded electrode from the exterior.

8. The pain signal measurement device as set forth in claim 1, further comprising:
a suction surrounding the microprobe array; and
an attaching controller deflating the suction to attach the microprobe array to the skin.

9. A pain signal measurement method of a pain signal measurement device including at least one microprobe array which includes a guard electrode disposed on a substrate; a plurality of microprobes penetrating the substrate and the guard electrode and electrically insulated from the guard electrode; and an insulating layer disposed between the guard electrode and a guarded electrode of each of the microprobes to reduce a noise between the microprobes, wherein a surface of the insulating layer of each of the microprobes is grounded to the guard electrode to be guarded by the insulating layer of each of the microprobes and the guard electrode, the pain signal measurement method comprising:
attaching the at least one microprobe array to the skin; and
making the at least one microprobe array penetrate the epidermis of the skin to measure an action potential of nerves distributed at the dermis of the skin.

10. The pain signal measurement method as set forth in claim 9, wherein making the at least one microprobe array penetrate the epidermis of the skin comprises:
deflating a suction surrounding the at least microprobe array.

11. The pain signal measurement method as set forth in claim 9, wherein the at least one microprobe array comprise a plurality of microprobe arrays.

12. The pain signal measurement method as set forth in claim 11, wherein at least one of the microprobe arrays is attached to a dorsal root ganglion to measure a pain signal, and another microprobe array is attached to an arm and a leg to measure a reference signal for determining the pain signal.

13. The pain signal measurement method as set forth in claim 12, further comprising:
estimating speed and position of the pain signal by using the reference signal.

14. The pain signal measurement method as set forth in claim 12, further comprising:
measuring the pain signal by varying arrangement and grounded shape of the at least one microprobe array.

15. The pain signal measurement method as set forth in claim 14, further comprising:
measuring a pain signal in the at least one microprobe array according to time; and
estimating a travel direction and an introduction direction of the pain signal from the pain signal measured according to time.

16. The pain signal measurement method as set forth in claim 12, further comprising:
distinguish pain patterns by using at least one of a cycle, generation time, an increasing rate, a decreasing rate, the intensity, and duration of the pain signal.

17. The pain signal measurement method as set forth in claim 12, further comprising:
mapping an acute pain or a chronic pain through the pain patterns.

18. A pain control method using a pain signal measurement device including at least one microprobe array which includes a guard electrode disposed on a substrate; a plurality of microprobes penetrating the substrate and the guard electrode and electrically insulated from the guard electrode; and an insulating layer disposed between the guard electrode and a guarded electrode of each of the microprobes to reduce a noise between the microprobes, wherein a surface of the insulating layer of each of the microprobes is grounded to the guard electrode to be guarded by the insulating layer of each of the microprobes and the guard electrode, the pain control method comprising:
making the least one microprobe array penetrate the epidermis of the skin to measure a pain signal from nerves distributed at the dermis of the skin;
transmitting the measured pain signal to an external circuit; and
making the external circuit transmit, to the at least one microprobe array, a pain control signal for modulating the pain signal based on the transmitted pain signal when the pain signal is transmitted to a brain.

19. The pain control method as set forth in claim 18, wherein the pain control signal has a phase of ±90 degrees to the pain signal, and wherein the cycle of the pain control signal is earlier than that of the pain signal.

20. The pain control method as set forth in claim 18, wherein the pain control signal is an AC or DC signal, and
wherein a pain is controlled by adjusting a phase difference of the pain control signal to the pain signal.
